(19) United States
(12) Patent Application Publication
(10) Pub. No.: US 2008/0031774 A1
(43) Pub. Date: Feb. 7, 2008

(54) Apparatus for Guiding Sample and Reagent Manipulations and Receptacles for Holding Same

(75) Inventors: Gary P. Magnant, Topsfield, MA (US); Michael J. Finney, San Francisco, CA (US); Todd J. Barbera, Marblehead, MA (US); George Corey, Newton, MA (US); T. Christian Boles, Bedford, MA (US)

Correspondence Address: BROMBERG & SUNSTEIN LLP, 125 SUMMER STREET, BOSTON, MA, 02110-1618, US

(73) Assignee: SAGE SCIENCE, INC., Beverly, MA (US)

(21) Appl. No.: 11/734,716

(22) Filed: Apr. 12, 2007

Related U.S. Application Data
(63) Continuation-in-part of application No. 11/685,557, filed on Mar. 13, 2007.
(60) Provisional application No. 60/791,390, filed on Apr. 12, 2006.
(60) Provisional application No. 60/781,895, filed on Mar. 13, 2006.
(60) Provisional application No. 60/798,917, filed on May 9, 2006.
(60) Provisional application No. 60/799,746, filed on May 11, 2006.
(60) Provisional application No. 60/890,689, filed on Feb. 20, 2007.
(60) Provisional application No. 60/858,542, filed on Nov. 13, 2006.

(51) Int. Cl. G01N 33/00

(52) U.S. Cl. 422/63; 422/100; 700/214

(57) ABSTRACT

A system has utility is guiding and tracking the execution of manual procedures. The system includes a support that holds multiple sample receptacles, computational circuitry to execute a protocol script that delivers instructions for executing various steps of the procedure, and a protocol progress sensor that advances the execution of the protocol script. The computer may be comparable in size to a support for holding the receptacles The receptacles be arranged in a frame, may be sealed with peelable or puncturable films and may include identifiers that are read by the system. The receptacles may be filled with a high heat capacity material to maintain a given temperature range. A variety of companion devices may interact with the system using the protocol script. An instrumented pipettor can communicate with the system for semi-automated execution of protocols.

### CROSS REFERENCE TO RELATED APPLICATIONS

This application is a continuation in part of U.S. patent application Ser. No. 11/685,557 for “Laboratory Reagent and Sample Assembly, Management and Processing”, filed Mar. 13, 2007 and thereby claims priority to that application and to the following United States Provisional patent applications:
Provisional application Ser. No. 60/781,895, filed Mar. 13, 2006 (Bromberg & Sunstein LLP attorney docket number 3094/102);
Provisional application Ser. No. 60/791,390, filed Apr. 12, 2006 (Bromberg & Sunstein LLP attorney docket number 3094/104);
Provisional application Ser. No. 60/798,917, filed May 9, 2006 (Bromberg & Sunstein LLP attorney docket number 3094/105);
Provisional application Ser. No. 60/799,746, filed May 11, 2006 (Bromberg & Sunstein LLP attorney docket number 3094/107);
Provisional application Ser. No. 60/890,689, filed Feb. 20, 2007 (Bromberg & Sunstein LLP attorney docket number 3094/112).
   This application also claims priority to provisional application Ser. No. 60/858,642, filed Nov. 13, 2006 (Bromberg & Sunstein LLP attorney docket number 3094/108). All of the above are hereby incorporated herein by reference.

### FIELD OF THE INVENTION

The invention generally relates to the packaging and use of reagent kits and the guiding of sample and reagent manipulations.

### BACKGROUND OF THE INVENTION

Scientists and technicians use a wide variety of instruments to produce chemical reactions in the laboratory. For example, a laboratory technician may produce a chemical reaction by dispensing reagents into one or more wells of a conventional microtiter plate. More specifically, as known by those skilled in the art, a microtiter plate typically has a flat portion with a two-dimensional array of independent wells that each form individual test tubes. Each well is capable of producing separate and distinct chemical reactions. One widely used type of microtiter plate has an 8×12 array of equally spaced wells. Accordingly, a lab technician may produce 96 separate chemical reactions in a single 8×12 microtiter plate.

Problems often arise, however, when the scientist, technician, or other laboratory personnel inadvertently dispenses fluid into the wrong well of a microtiter plate, or dispenses the wrong fluid or dispenses fluid at the wrong time. Among other undesirable results, these types of human error (or machine error in an automated process) can produce incorrect test results, waste time and resources, and increase the overall cost of the experiment.

As a further inconvenience to the user, reagent kits often lack needed components. Kit manufacturers often require users to supply certain components due to differences in storage conditions among reagents. Reagents may come from different lots that are exposed to different storage conditions and components may be inactive due to environmental conditions or expiry. Certain reagents may also be unstable at room temperature and require chilling in a ice bath or cooler that is separate from the reaction plate.

### SUMMARY OF THE INVENTION

In accordance with an illustrative embodiment of the invention, a system aids in the guiding of reagent manipulations. The system includes a support that is adapted to hold a plurality of sample receptacles and a computer that is adapted to execute a protocol script so as to present a first instruction to an experimentalist. The computer is sized to be comparable to the support. The system also includes a protocol progress sensor for causing the computer to advance the script to present a second instruction in response to a given degree of protocol progress.

The computer may reversibly attach to the support as a base or a lid to the support and may be clipably attachable to the support.

In another embodiment of the invention, there is a system for storing and assembling a plurality of reagents that includes a frame with a microplate footprint and a plurality of differently sized or shaped cavities, and a plurality of inserts sized to be inserted into the plurality of cavities. A least one insert has at least one fluid receiving member.

Optionally or additionally, an insert is sealed with a, puncturable film, an insert is sealed with a peelable film, or an insert is sealed with a puncturable film and a peelable film, with the peelable film adhered atop the puncturable film. A fluid receiving member may have an outer layer, an inner layer, and a high heat capacity material therebetween.

The high heat capacity material may be, for example, a gel or a liquid, and may change phase at a predetermined temperature, such as 4° C. The space between the outer layer and the inner layer may be at a sub-atmospheric pressure.

Optionally or in addition, at least one insert is an analytical device.

In a related embodiment the system includes a protocol or a pointer to a protocol including a series of instructions associated with reagent or sample manipulation steps of a procedure to be implemented by an experimentalist. The protocol is in a format readable by a computerized apparatus adapted to accept input related to a previously performed step and to use the input to select an instruction for the user as to how to perform a future step.

Optionally or in addition, at least one insert includes machine readable data related to the contents of the fluid receiving member therein. The data may relate to reagent identity, lot number, production date, expiry date, or storage conditions, reagent concentration or specific activity.

Optionally or in addition, an insert comprises a feature for facile removal from the frame. The feature may be for example, a hole, a tab, a pin, a lip or a flap.

In accordance with another embodiment of the invention there is a system for guiding reagent manipulations that includes a support adapted to hold a frame having a plurality of differently sized or shaped cavities for holding a plurality of inserts sized to be inserted into the plurality of cavities. The system also includes a computer adapted to execute a protocol script so as to present a first instruction to an experimentalist, and a protocol progress sensor for causing the computer to advance the script so as to present a second instruction in response to a given degree of protocol progress. The support includes a reader for receiving machine-readable data that is physically associated with the receptacle.

The reader may include, for example, a wired connector or an antenna for receiving a wireless signal.

In accordance with another embodiment, there is a method for manufacturing a kit. The method includes associating a reagent with a protocol script, packaging the reagent in an insert adapted to mate with a frame that has a complementary cavity, and providing the reagent and the script to an experimentalist.

Optionally, or in addition, the method includes providing the frame to the experimentalist.

In accordance with another embodiment, there is a method for performing a reaction that includes the steps of providing a frame having cavities, inserting the frame into a support having at least one sensor adapted to provide information related to the degree of completion of a sample manipulation procedure, inserting an insert into the frame, the insert having a fluid retaining member, receiving an instruction from a computer running a protocol script; removing a liquid from the fluid retaining member, and transferring the liquid to a second fluid retaining member.

The step of inserting an insert into the frame is performed prior to or after the step of inserting the frame into the support.

In accordance with another embodiment of the invention there is a system for use in semi-automated liquid transfer procedures that includes a support that is adapted to hold a plurality of sample receptacles, and a computer that is adapted to execute a protocol script so as to present a first instruction to an experimentalist. There is also a protocol progress sensor adapted to cause the computer to advance the script so as to present a second instruction in response to a given degree of protocol progress. The system also includes an instrumented pipettor that has a programmably adjustable volume setting and a wireless communication assembly. The pipettor transfers records of liquid handling events to the computer.

Optionally or in addition, the protocol progress sensor sends a signal to the pipettor in response to detecting a well penetration event. The protocol progress sensor may detect an occlusion of a light source.

In accordance with another embodiment of the invention there is a system for use in semi-automated liquid transfer procedures that includes a support that is adapted to hold a plurality of sample receptacles, a computer that is adapted to execute a protocol script so as to present a first instruction to an experimentalist, a protocol progress sensor adapted to cause the computer to advance the script to present a second instruction in response to a given degree of protocol progress, and an instrumented pipettor that has a programmably adjustable volume settings and a wireless communication assembly. The protocol progress sensor actuates the pipettor to perform an action selected from the group consisting of adjusting a pipettor volume setting, actuating liquid withdrawal, or actuating liquid dispensing.

Optionally or in addition, the protocol progress sensor sends a signal to the pipettor in response to detecting a well penetration event. The protocol progress sensor may operate to detect an occlusion of a light source.

### BRIEF DESCRIPTION OF THE DRAWINGS

The foregoing advantages of the invention will be appreciated more fully from the following further description thereof with reference to the accompanying drawings. Below is a brief description of the drawings.

FIG. 1 is a block diagram showing components of a system for guiding and tracking the manipulation of samples and reagents;

FIG. 2 schematically shows a perspective, partially cut-away view of fluid mixing apparatus and system configured in accordance with illustrative embodiments of the invention;

FIG. 3 schematically shows a plan view of the fluid mixing apparatus and system shown in FIG. 2;

FIG. 4 schematically shows a method of the fluid mixing apparatus and system shown in FIG. 2;

FIG. 5 schematically shows a plan view of the fluid mixing apparatus and system of FIG. 2 illuminating a first set of wells;

FIG. 6 schematically shows a plan view of the fluid mixing apparatus and system of FIG. 2 illuminating a second set of wells;

FIG. 7 schematically shows a perspective view of an alternative system for mixing fluids having an external display device;

FIG. 8 schematically shows another alternative system for mixing fluids having two systems that each have a support member and receptacle;

FIG. 9 shows a perspective view of a support member in assembled configuration;

FIG. 10 shows an exploded view of the support member;

FIG. 11 shows a top view of a microplate sized support member, a sub-microplate support member, and a sub-microplate receptacle;

FIG. 12 shows a perspective view of an interactive benchtop containing multiple wirelessly networked laboratory devices;

FIG. 13 shows a support member with a computer of comparable size supported on a stand;

FIG. 14 shows a support member with a computer of comparable size suspended with a clip;

FIG. 15 shows a disassembled perspective view of a frame and receptacles that mate with the frame;

FIG. 16 shows a partially assembled perspective view of the frame and receptacles of FIG. 16;

FIG. 17 shows a frame with transparent receptacles;

FIG. 18 shows a microplate format and sub-microplate format frames with multiple sealed and unsealed receptacles;

FIG. 19 shows a perspective view frame and receptacles being used with a support;

FIG. 20 shows a sectional view of a receptacle with a high heat capacity material between an outer layer and an inner layer; and

FIG. 21 is a flow chart for a semi-automated liquid handling procedure in accordance with an embodiment employing an instrumented pipettor.

### DESCRIPTION OF ILLUSTRATIVE EMBODIMENTS

In illustrative embodiments of the invention, a system guides an experimentalist in executing a procedure, records data related to steps in a procedure for purposes of altering subsequent steps in the procedure, annotating, verifying the results of the experiment, generating reports, and aiding in the analysis of data generated during the experiment. The system typically includes computer logic and associated peripheral devices that collect data regarding experimental steps and transmit the data to the computer. Examples of suitable peripheral devices include without limitation: devices for tracking the movement or actuation of a pipettor, ejection of a pipette tip, measuring humidity, temperature, cell growth, conductivity, pH, pIon (e.g., pCa++), level, liquid volumes, light or volatile organics, global position, bar code readers, biometric identification readers, voice recorders, video recorders, sensors that detect introduction of samples (including solids or liquids) into a receptacle or analytical tool, and analytical tools that return data regarding settings, run conditions and results. The computer records the data at one or more time points in the experiment for later use. After completion of experimental steps, the system may generate a report that includes or utilizes the acquired data. The system may acquire external data from a networked analytical tool and combine the external data with data from at least one integral sensor to calculate a parameter (e.g., a reaction rate). Other aspects of the invention relate to devices and methods for packaging and assembling reagent kits, including kits that interact with components that guide or record various procedural steps.

The system may also instruct a user on various steps of the experimental process or protocol; as a result, the productivity of the user may increase and the user may more readily learn new techniques. The instructions given may be dynamic; the instructions for a given step may change based on data received from the various companion devices, consumables, or database lookup steps. Instructions may be given in various forms including, without limitation, recorded voice instructions, computer-generated voice instruction, non-voice audible signals (e.g., tones, chimes, buzzing), visual instructions including icons or text displayed on an electronic display and illumination of LEDs.

In illustrative embodiments, a system detects when a fluid transfer apparatus (e.g., a pipette) is positioned to transfer fluid to or from the well of a receptacle (e.g., a microtiter plate) it supports. Detecting the fluid transfer apparatus in this manner consequently enables a variety of other useful functionalities. Among others, the fluid handling system may responsively guide the user through a complete dispensing process, record actual results of the process, and elicit an audible or visual alarm if the user attempts to transfer fluid to or from the wrong well, or at the wrong time. Details of this and other embodiments are discussed below. As a result, it is anticipated that fewer procedural errors will be made, thereby saving expense and reducing waste, including hazardous waste.

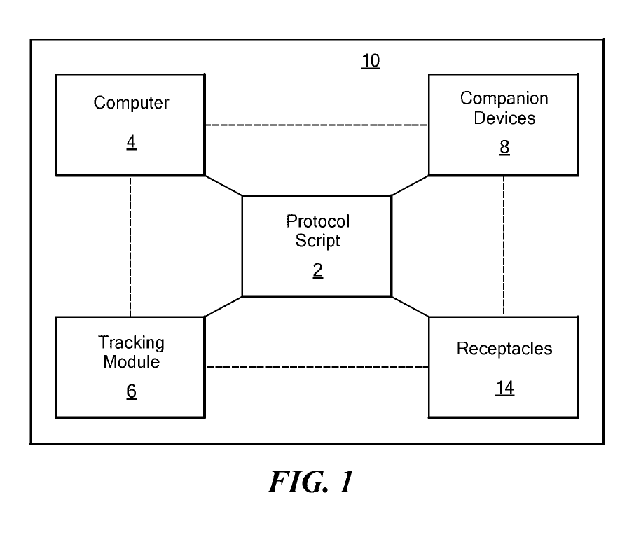

FIG. 1 is a block diagram showing various components of a system 10 for guiding sample and reagent manipulations. A protocol script 2 contains a list of instructions for executing a laboratory procedure. The procedure may be, for example, a research, diagnostic, forensic, sample collection, compound library procedure (e.g., formatting, daughter plating, cherry picking), or other procedure. The protocol script is executed in conjunction with a laboratory device network that may include, among others, a computer 4 (including one or more displays), a sample/reagent manipulation tracking module 6 that records sample manipulation steps including dispensing and withdrawing of fluid or solid samples, and one or more companion devices 8 that interact with the protocol 2. The protocol script 2 instructions may be manifested by the system 10 in a variety of ways, including through text and graphical displays, illumination of LEDs, audible alerts or messages, electronic communications (e.g., email, text messaging, and machine signals to other components). In embodiments, the protocol script may be associated with a particular experiment, a particular peripheral device, or reagent kit through programming by a user or manufacturer. Protocol scripts 2 may be physically bundled with reagent kits in machine-readable form (e.g. a compact disc, DVD, RFID chip, flash memory device (including a USB memory card), either directly, or as a machine-readable identifier that points to a protocol stored on a network (e.g., on the computer 2, or downloadable via the internet).

The computer 4 may be any of a variety of information processing devices, housed in various formats, including a desktop personal computer, a laptop/notebook, sub-notebook, personal digital assistant. As discussed below, the computer 4 may be a device sized to fit unobtrusively on a laboratory benchtop. In various embodiments discussed herein, computing functions may be distributed across various components, including the tracking module 6, companion devices 8 or others. The computer 4 may store a protocol script, present instructions to the experimentalist for the multiple procedural steps embodied in the protocol script, receive information related to the degree of completion of the procedure and update a pointer, and otherwise accept and relay various communications among the various system components. The computer 4, or other system components (including the tracking module 6) may have various input/output assemblies and functionalities, including an associated wireless communication receivers or transceivers that operate using various modalities; for example infrared, radio wavelength, magnetic, including RFID, Zigbee, RuBee, bar-code reader or the like, along with associated circuitry, antennae, etc . . .

The tracking module 6 is a protocol progress sensor that serves the function of detecting when a step in the protocol script 2 has been attempted or successfully completed. In a simple form, the tracking module 6, could comprise a manual advance button such as a mouse button or foot pedal that is actuated by a user after completion of a sample or reagent handling step. In more elaborate examples, the tracking module 6 uses one or more sensors to detect pipettor proximity to one or more fluid receiving members of receptacles 14 (e.g., microplate wells, reagent troughs, microtubes, analytical device sample ports etc.) The tracking module 6 may detect and record when a tip of a dispensing tool (hereinafter exemplified by a pipette tip) has penetrated or otherwise reached a threshold proximity to a fluid receiving member or a receptacle 14 or portion thereof. For example, the sensors may triangulate the position of the pipette tip using sonic, electromagnetic, or other signals, or, as described below, may be optical assemblies positioned on a support to be adjacent to opening of wells in a microplate receptacle 12 so as to detect penetration of the wells.

Optionally, or additionally, the tracking module may detect liquid withdrawal or dispensing events as well as other data associated with those events (e.g., volume settings), that may be transferred by the pipettor or automated liquid dispenser to the computer.

In an embodiment, for optimal minimization of benchtop clutter, the computer 2 is sized to be comparable to the tracking module, and one or both may be wireless. The so-sized computer 2, described in more detail below is referred to herein as a “compact data processing apparatus.”

The companion devices 8 may be any of a variety of laboratory devices including, without limitation, a handheld pipette, a controlled temperature block, a robotic liquid dispenser, an optical microplate reader, an electrophoresis assembly, a microplate autosampler (as might be used in high throughput HPLC, LC-MS, NMR, etc). The companion device is part of the system network, and may include wireless communication circuitry for communicating with other system components, including other companion devices 8. In an embodiment, a handheld pipette includes mechanisms for automatic volume setting, actuation, and wireless communication with the system 10.

The receptacles 14 may include, for example, microplates, microstip wells, reagent troughs, microtubes, or, in an embodiment described below, collections of multiple such receptacles 12.

The system 10 may include a receptacle support, which may function as a positioning jig, which in embodiments is integral or identical to the tracking module 6. The system support may also house circuitry and other components for assisting an experimentalist with performing a procedure. For example, the support may include, in various combinations, a microprocessor, wireless communication circuitry, a computer display (e.g., an LCD, or a touch-screen LCD), user input buttons, a battery, a temperature controller (e.g., for executing time-temperature profiles, constant temperatures, creating multiple zones at different temperatures, or temperature gradients), a loudspeaker, well indicia, a clock/timer, an electrophoresis power supply, a headphone jack, a joystick, a hard drive, a flash memory, a buzzer alarm, digital audio and video circuitry (e.g., for MP3, DIVX, VOIP), etc.

The support 12, computer 4, or other components may be battery powered. Alternately or in addition, they may include inductive charging receivers, and may thereby be more highly sealed and resistant to liquids. Various components may include ports for wired communication connection; for example, USB, Firewire™, or may have universal connectors for connecting to a dedicated adapter or docking station.

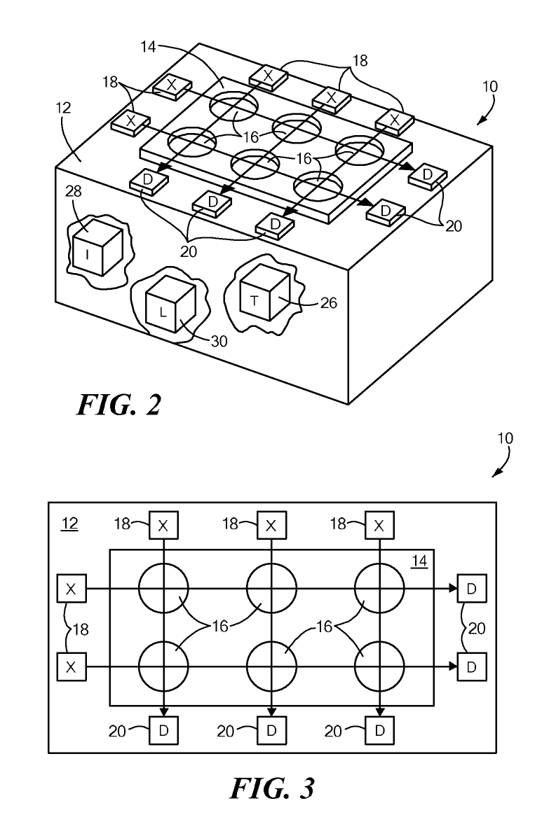

FIG. 2 schematically shows a perspective, partially cut-away view of a fluid-tracking module 6 of a system 10 configured in accordance with illustrative embodiments of the invention. FIG. 3 schematically shows a plan view of the same system 10. The system 10 includes a support member 12 configured to support a receptacle 14 having at least one well 16. The support member 12 may be configured to have a central recess that receives the receptacle 14. In accordance with illustrative embodiments, the support member 12 also has detection components for detecting access of a supported receptacle 14 (discussed in detail below).

The receptacle 14 may be a microtiter plate (also identified herein by reference number 14) having a plurality of wells 16. Although only six wells 16 are shown (a 3×2 array), various aspects of the invention apply to use with microtiter plates having fewer or more wells 16. For example, the microtiter plate 14 can have a single well, 24 wells, 96 wells, 384 wells, or up to 1536 wells, or more wells. In illustrative embodiments, the microtiter plate 14 used with the described support member 12 is a standard, off the shelf component having 12 columns and 8 rows of wells 16. Although not discussed below, in an alternative embodiment, the plates 14 are not standard; instead, such plates 14 have integrated detection components.

For simplicity, this discussion refers to the receptacle 14 as a conventional 3 column, 2-row microtiter plate 14. It should be noted, however, that discussion of the receptacle 14 as a microtiter plate 14 is for illustrative purposes and thus, is not intended to limit all embodiments of the invention. Accordingly, those skilled in the art should understand that other receptacles 12 can be used, such as single or multiple microfuge tube(s), trough(s), or test tube(s). Additionally, in embodiments described below, multiple formats may be arranged in a frame, which, optionally, has a microplate footprint and fits into the support member 12.

As noted above, the support member 12 has a plurality of detectors that detect the presence of a fluid dispensing apparatus (e.g., a pipette) when such apparatus is in a prespecified position for adding fluid to, or withdrawing from, any of the wells 16. To that end, the support member 12 of FIG. 2 has one row of three light emitting diodes 18 (transmitters), and one row of three corresponding light detectors 20. In a like manner, the support member 12 also has one column of two light emitting diodes 18, and one column of two corresponding light detectors 20. Each light emitting diode 18 generates a light beam that is directed in a substantially straight line toward its corresponding detector 20. Accordingly, because the diodes 18 and detectors 20 are arranged in an array format, each well 16 of a supported receptacle 14 has two substantially orthogonal light beams positioned in relatively close proximity to its opening. As discussed below, these two orthogonal light beams effectively form a cross-hair preferably having a center substantially at the center of each well opening.

In some embodiments, the support member 12 may be preconfigured so that it can accept a specific type of receptacle 14, such as a standard 8×12 microtiter plate. Accordingly, the diodes 18 and detectors 20 are pre-positioned to produce an array of cross-hairs 24 that, as shown in the figures, align with the array of well openings of that specific plate 14. In fact, the diodes 18 form these cross-hairs even when the support member 12 does not support the plate 14. The support member 12 therefore also preferably has a jig that precisely positions the supported plate 14 to ensure proper alignment with the cross-hairs 24. In alternative embodiments, however, the detectors 20 are movable to accommodate varying sized receptacles. The support member 12 is generally made for use with a microplate 14 having a maximum rated size. In such case, the support member 12 can be used with receptacles 14 having fewer wells 16. For example, a 96 well plate 14 may be used with a support member 12 designed to also accept a 384 well plate 12.

Some embodiments can use motion or other proximity detecting technology other than the diodes 18 and detectors 20 shown in FIG. 2. For example, each well 16 may have a single motion-detecting device. In other embodiments, the light sources 18 may be laser diodes or infrared lasers. For embodiments in which each light source contributes to cross-hairs above multiple wells, lasers have an advantage of being highly collimated. Accordingly, in that case, the cross-hairs are formed from beams of approximately uniform width throughout the illuminated plane. Non-coherent light sources, including light emitting diodes 18, may be coupled with collimating optics to improve their performance. The light sources may include light guiding mechanisms such as fiber optics or mirrors (especially in combination with lasers) that allow light sources 18 to be mounted remotely, yet still illuminate the proximity of the wells for detection by the detectors 18. In a related embodiment, a flash lamp built into the support 12 is used to excite fluorescence; an optional cover may be used to decrease stray light and the cover may be mirrored to increase sensitivity.

To increase reliability, redundant light sources and detectors 20 also may be used. For example, the system may have cross-hairs in two or more parallel planes, or in side-by-side arrangement. A system having redundant cross-hairs may advantageously have the diodes 18 and detectors 20 in an alternating configuration to compensate for sub-optimal light collimation. If the redundant cross-hairs are in two parallel planes, then the system may be configured to recognize a dispensing event only if both cross-hairs above a given well are disrupted. Of course, the detectors 20 use corresponding detecting technology suitable to detect the illuminating wavelength and intensity. In another reliability-increasing embodiment, the system does not register a penetration event unless the light source is blocked for a predetermined threshold time.

Some embodiments integrate a visual display (not shown) into the support member 12 to provide additional instructions and data (e.g., data showing the volume of liquid to be added or removed) in real-time. Moreover, as discussed above, the support member 12 also may contain a number of other functional elements that cooperate with the wells 16 of the receptacle 14, and the diodes 18 and detectors 20. Those functional elements (shown in a cut-away portion of the support member 12 in FIG. 2) include a temperature module 26 for controlling the temperature within the wells 16 of a supported receptacle 14, and an optical measurement apparatus 28 for capturing optical properties, such as UV/visible absorbance, fluorescence, or luminescence of samples within each well 16. The functional elements also include a logic module 30 for coordinating, among other things, the diodes 18, detectors 20, optical measurement apparatus 28, and temperature module 26. The logic module 30 also may coordinate with external components, such as external computers and memory. In alternative embodiments, the logic module 30 is an external computer system, server, or other similar logic device. Details of the interaction of these functional elements are described in greater detail below. The temperature module 26 may be configured to have multiple temperature zones to, for example, keep certain wells cold (e.g., 4° C.) to prevent degradation of contents therein and/or to keep other wells warm (e.g. 37° C.) to encourage biochemical reactions.

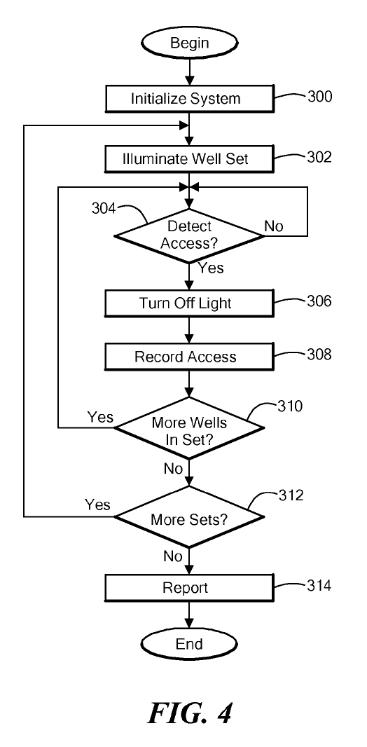

FIG. 4 schematically shows a method of assisting the fluid mixing process in the system shown in FIG. 2. The process begins at step 300, which initializes the system. To that end, among other things, a user may place a receptacle 14 (e.g., a microtiter plate) on the support member 12 before, during, or after the logic module 30 begins its initialization processes. Specifically, among other things, the logic module 30 may initialize the system 10 by energizing the light emitting diodes 18 and detectors 20, and initializing communication with external devices, such as a computer and memory device. The logic module 30 also may control a graphical user-interface for controlling other functional elements, such as the temperature module 26 and an optical measurement apparatus 28. Alternatively, the logic module 30 may be preprogrammed to control the optical measurement apparatus 28 and temperature module 26 in accordance with a prescribed set of instructions. In addition, the logic module 30 may create a record in memory for storing details of the mixing process, and assign wells 16 to a plurality of different well sets (discussed below).

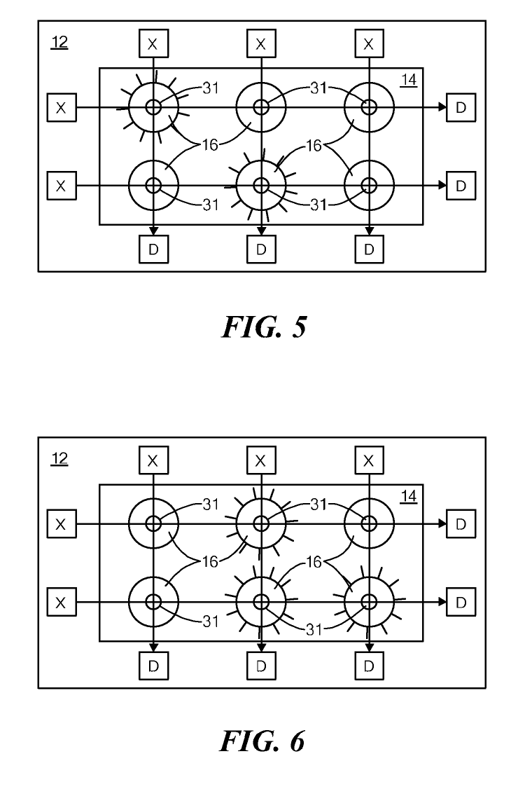

The process then continues to step 302, which illuminates a first set of wells 16 (i.e., a prescribed group having between zero to six wells 16). To that end, each well 16 in the microtiter plate 14 shown in FIG. 2 has one or more associated light elements 31 that may be selectively illuminated for a specific purpose (see FIG. 5). For example, the light elements 31 of the top left and bottom center wells 16 of FIG. 5 are illuminated.

The meaning assigned to an illuminated well 16 may vary depending upon application. For example, an illuminated well 16 may indicate that the reaction in that well 16 is complete, or that a reagent should be removed from that well 16. As another example, an illuminated well 16 may serve as a visual indication that the user should add a reagent to that particular well 16. As discussed below, information to the experimentalist may be manifest in the color of the illumination, or through the flashing of the indicia at a given rate. In some embodiments, illumination of a set of wells may be accompanied by real-time instructions displayed on an associated display device. The instructions may indicate, among others, the type and volume of reagent to be added or removed from one or more wells 16. As discussed below, if an instrumented pipettor is used, the system 10 may communicate with the pipettor to adjust an aspirating or dispensing volume automatically

The light elements 31 for each well 16 may be any of a variety of colors. For example, a green light may indicate that reagent should be added, a blue light may indicate the wells involved in an imminent step, and a red light may indicate an error condition (for example, when a reagent is being or has been added to the wrong well or at the wrong time). Accordingly, a single light element may be capable of emitting multiple colors, or comprise multiple different light elements having different qualities. Visual indicia also may be provided to indicate whether a volume is to be added or removed during a particular fluid handling step. Additional qualities of the emitted light can be modified, such as the intensity of the light, and whether the light element 31 is blinking. By combining 2 differently colored light sources and cyclically flashing them on and off for controlled periods of time at a cycle frequency that is faster than can be perceived by the human eye, additional colors may be simulated. For example, relatively inexpensive red and green LEDs may be used as light elements 31; by alternately flashing them at a frequency of greater than about 10 Hz, simulated yellow or orange colors may appear. Colors may be varied by altering the relative on-time for each LED in the cycle. Similarly, using three colors of LEDs can enable an even broader range of colors.

To that end, in illustrative embodiments, an LED control can use 3-state CMOS drivers with a VDD of, for example, 5 volts. In fact, among other ways, all LEDs can be independently controllable, using a single output line per LED, as follows:
Red/Green LEDs coupled in antiparallel (e.g., on a single device), with one leg connected through a resistor to a rail near 2.5 volts, and the other leg connected to a 3-state output. If the output is five volts, it is red, if zero volts, it is green, 3-state is off, and various square waves give different colors. The 2.5 volt rail actually may be adjusted to a voltage that will equalize the intensity of red and green.

After the first set of well light elements 31 are illuminated, the process determines if the user has accessed any of the wells 16 (step 304). To that end, the process determines if any of the light beams have experienced any mechanical interference. Specifically, if the user positions a pipette near the opening of a well 16, it may intersect substantially with the associated well cross-hair. At this point in the process, the user may dispense or withdraw fluid from the well 16 in a conventional manner. If the pipette is not positioned close enough to the well opening, however, fluid ejected from the pipette may necessarily intersect the cross-hair, thus providing the requisite detection function.

Interference with the signal may be manifested in the form of a voltage change or other signal from the appropriate row and column detectors 20. The associated row and column detector pair thus transmit such signals to the logic module 30 indicating that something has interfered with their respective received light signals. Although a number of mechanical objects can break a light beam, it is recommended that users ensure that only a pipette or other fluid transfer apparatus does, in fact, break the light beam. To reduce the chance of a spurious triggering decision, additional correlative data may be incorporated into the decision; for example, triggering sensitivity may be temporarily increased around the time that a pipetting step is instructed by the protocol script, or when a RFID tag affixed to a pipetting instrument's is detected by the support member 12. A predetermined dwell time threshold may be required; e.g., a detector 20 voltage drop below a threshold for more than 1 second.

In the event that a user inadvertently triggers one or more detectors 20, an over-ride routine may be provided to allow normal operation to resume. The over-ride may be logged by the computer for attachment to the data set. In any event, it is important for the cross-hair to be substantially concentrically positioned over the center of the well opening. Although not optimal, however, off-center cross-hairs 24 still should suffice in many instances.

In a similar manner, illustrative embodiments position the cross-hair a very small distance above the plane of the well opening. For example, empirical tests may show approximate distances that users position a pipette from the opening when transferring fluid into or from a well 16. The cross-hair therefore may be positioned substantially at that height, or some distance slightly above that height. Care should be taken that the cross-hairs not be positioned so close to the well openings that normal variation or warping of microplates 14 will break the light beams. In some embodiments, however, the two substantially perpendicular beams forming a cross-hair do not physically intersect. Specifically, in that case, the beams may be on separate planes and thus, not directly interfere.

The detectors 20 are considered to detect when an instrument penetrates/accesses the plane of a specific well opening (i.e., the plane formed by the well opening) if it detects interference of the cross-hair associated with that well 16. Such interference may be any change in the integrity of the cross-hair. For example, such interference could be a direct blocking of the beam or simply a reduction in light intensity.

After it detects penetration/access in the first well 16 and determines that such well 16 is in the first set, the logic module 30 turns off the light associated with the first well 16 (step 306). In alternative embodiments, the light element 31 may remain on, change color, or blink at some prescribed frequency. The logic module 30 may sound an alarm if the well 16 is not in the first set.

The process then continues to step 308, in which the logic module 30 records the date and time of access of the specific well 16 just accessed. As suggested above, this record could be maintained in a local memory device, or transmitted externally, such as by a wired or wireless connection, to an external memory device. The memory device may include either or both volatile or nonvolatile memory, such as a hard disk drive or disk array implementing a fault tolerant memory protocol (e.g., RAID).

Among other things, the record may be associated with an experimental data set in a laboratory information management system (LIMS). A bar code or RFID reader also may be incorporated into the system 10 to assist in tracking the data. Of course, the logic module 30 may also cause recordal of data other than well penetration data. For example, the logic module 30 may record environmental conditions, such as microtiter plate temperature. When operated in conjunction with an electronic liquid dispenser adapted for data communication with the computer (e.g., a handheld automatic pipette with a wireless transceiver), such as liquid handling robot or semi-manual instrumented pipette, the system 10 may record the volume setting of the dispensing instrument. These various records may be useful in confirming the accuracy of a procedure for various reasons, including scientific and forensic purposes (e.g., use as evidence in a criminal trial). The logic module 30 also may record the time or times at which reactions were started or stopped in multiple wells. The start times may then be subtracted from the times at which a reaction was stopped or analyzed to compute a reaction rate or otherwise normalize reaction data for variations in reaction time. For example, a microplate reader may measure a fluorogenic signal and a background reading; a computer may subtract the background reading from the signal and then divide by an automatically measured reaction incubation time to arrive at a reaction rate. Since reactions proceed at varying rates depending on temperature, the system may also apply corrections to rate data to normalize for temperature variations; the corrections may be based, without limitation, on an average temperature or a temperature integrated over an incubation time.

Step 310 then determines if the set of wells 16 currently being processed has more lit, unaccessed wells 16. If the set has more illuminated, unaccessed wells 16, then the process loops back to step 304 to monitor the other wells 16. Conversely, if step 310 determines that the set has no additional lit, unaccessed wells 16, then the process continues to step 312, which determines if the process has any more sets of wells 16 to be processed.

If there are additional sets to be processed, the process loops back to 302, which illuminates the next set of wells 16. FIG. 5 schematically shows a second set of wells 16 of the same microtiter plate 14 that may be processed as discussed. It should be noted that the second set has a different number of wells 16 than that of the first set, while both sets share a common well 16. Of course, both sets could have no common wells 16, or all common wells 16.

Returning to step 312, if there are no additional sets to be processed, then the process continues to step 314, which generates a report summarizing the actual steps (i.e., well accesses) taken by the user. This report may be either hard copy or computer copy, and may be used as a record of the entire experiment.

It should be noted that application of the well detecting technology discussed in FIG. 4 is illustrative. Other processes may be used, such as a simple detecting and recording process that does not illuminate the wells 16 (e.g., creating a record or protocol of the experiment), a triangulation process using multiple sensors, or a process that simply illuminates one well 16 at a time (i.e., each set having one well 16 only). As another example, the process simply may responsively illuminate the wells 16 on a column by column basis, or on a row by row basis.

As noted, the process of FIG. 4 may be modified to also alert the user when a well 16 has been erroneously accessed. To that end, when a pipette breaks the cross-hair of a well 16 that is not in the set currently being processed, the logic module 30 may illuminate LED light elements 31 of a particular color (e.g., red), generate an error message on a user interface, or actuate an audible alarm signal. As suggested above, this modified process may be used with or without lighting wells 16 in the set, and/or without recording the results. It nevertheless is preferred that the logic module 30 record all results, whether or not they indicate erroneous well accesses.

An embodiment of the invention further increases laboratory worker productivity and increases the probability of a successful experiment. After recording a start-time, the logic-module 30 may start a timer and trigger one or more alarms after a preset elapsed time to remind a user to perform an additional step, such as adding an additional reagent to particular wells 16, or recording the results of a reaction with a microplate reader; a user can thus more readily perform additional tasks during incubation steps without the risk of forgetting about the reaction occurring in the wells 16. The alarm can be any of a variety of audible or visual signals, a vibration, a computer generated or processed voice, an email, page, text message or any of a variety of other suitable signals. The message may be nonspecific (e.g., a loud beep), or give specific details such as how much of a particular reagent to add at a specific time. For example, a computer generated voice may announce “Alert: add ten microliters of one molar sodium hydroxide to plate number one in three minutes.” Repeat reminders may be provided and the system 10 may include a snooze feature to defer future reminders for a given period of time.

The temperature module 26 and an optical measurement apparatus 28 may be used before, during, or after the process of FIG. 4, and have the functionality of a temperature-controlled optical microplate reader. For example, the an optical measurement apparatus 28 may acquire optical information, such as the fluorescently measured reaction progress of a sample in a given well 16 before and after a reagent is added. The optical measurement apparatus may be either intrinsic to, external to, or attachable to the support 12. In some embodiments having an optical measurement apparatus 28, the light elements 31 can function not only as visual signals to a user, but to excite fluorescent transitions of molecules in the wells 16 for quantitative or qualitative analytical purposes. If used as excitation elements, the light elements 31 may be equipped with filters to reduce stray light, and/or to allow fluorescently multiplexed assays. Detection components may be placed under the wells 16 or over the wells 16 (e.g., the system 10 could be placed under an array of photodetectors such as a CCD array or an array of independent detectors). As with the illumination optics, the detectors may be remotely placed if the system is equipped with suitable light guides such as optical fibers or mirrors. If fluorescence is to detected, suitable excitation optics are provided In an illustrative application, the an optical measurement apparatus 28 may be used to determine the quantity of DNA in each well 16 if the DNA is bound to a fluorogenic reporting reagent such as SYBR Green (Invitrogen Corporation, Carlsbad, Calif.); the presence or absence of the proper amount of DNA may be communicated to a user during or after completion of the procedure.

The temperature module 26 may keep a reagent or mixture of reagents cool (e.g. to 4° C.) to prevent unwanted reactions. Upon detecting that a start reagent has been added to all of the desired wells, the temperature module 26 may automatically increase the temperature to some desired temperature (e.g. to 37° C.). Each optical data point may be transferred electronically to an external computer system for subsequent processing. In a similar manner, the temperature module 26 may be manually or automatically activated at specific times to heat or cool specified wells 16. For example, the temperature module 26 may automatically increase the temperature of a given well 16 after the logic module 30 detects that a pipette has added a reagent to that well 16. The temperature module 26 is programmable and may carry out a programmed time-temperature profile and may create precise temporal and spatial temperature gradients.

As another example, various embodiments may compute reaction rates by means of time of reaction data and data from the optical measurement apparatus 28. This feature is especially useful for rapid reactions. Still another use of the optical measurement apparatus 28 may be as a confirmation of the well penetration data from the detectors 20.

Of course, the temperature module 26 and optical measurement apparatus 28 may be used in wide variety of additional applications. Accordingly, the specific applications discussed above are mentioned for exemplary purposes only and thus, are not intended to limit all aspects of the invention.

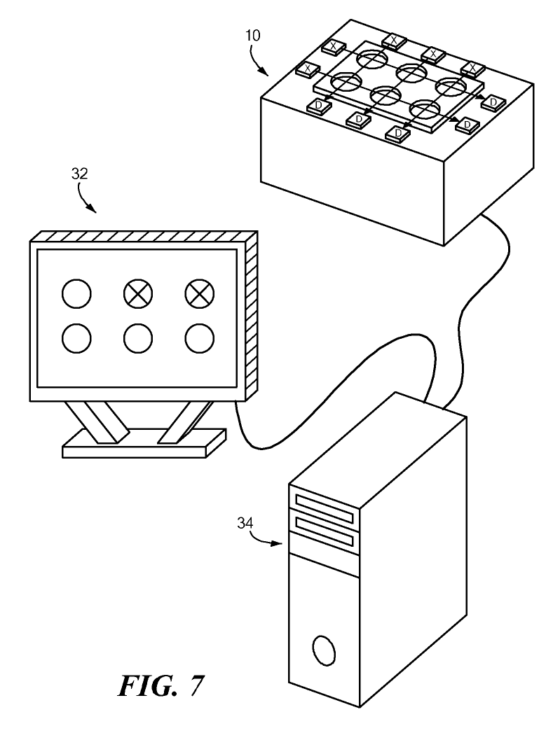

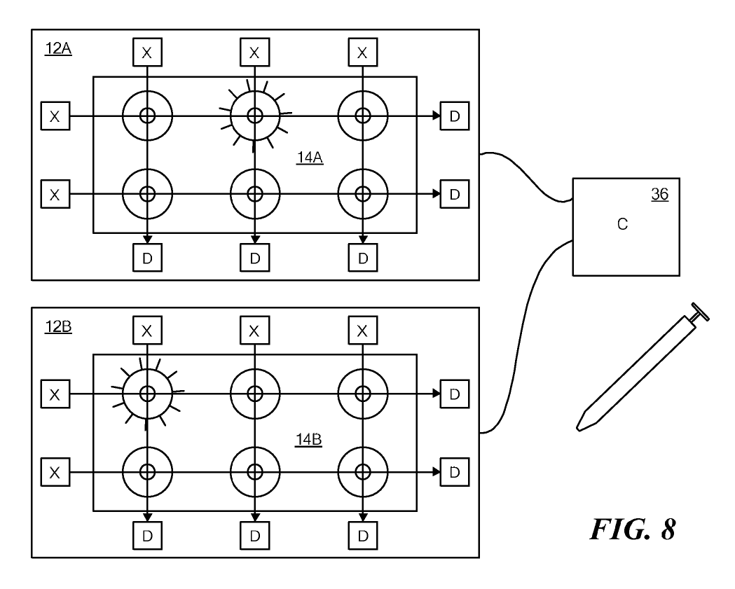

As noted above, some embodiments use an external display, such as liquid crystal display (LCD) device, a cathode ray tube display device, and/or touch sensitive screen, as a user interface. Such embodiments also may use the external display in addition to, or instead of, the lights illuminating each well 16. FIG. 7 schematically shows such an embodiment. In particular, the embodiment in FIG. 7 shows the system 10 of FIG. 2 connected to a liquid crystal display device 32 and a computer system 34. Accordingly, the liquid crystal display device 32 may implement a user interface, and highlight specific wells 16. As shown by example, the system 10 shown in FIG. 8 is processing a set that includes at least the top center and top right wells 16. If an LCD is positioned under the receptacle 14 and used to illuminate specific wells 16, an additional portion of the LCD may be used to communicate procedural instructions to the experimentalist.

A display device, such as the liquid crystal display device 32 of FIG. 7, may be positioned in a support underneath a clear microtiter plate and thus, serve the same function as the visible light elements 31. Of course, placing light elements 31 (e.g., light emitting diodes) or a liquid crystal display device 32 underneath the microplate 14 is of little use if the microtiter plate 14 is opaque, as is commonly the case when measuring fluorogenic reactions. In this case, some embodiments position the light elements 31 in-line with each column or row to indicate which wells of the microtiter plate 14 should be accessed for liquid dispensing and removal.

FIG. 8 shows another embodiment of the invention using a plurality of microtiter plates 14. Specifically, the embodiment shown in FIG. 8 has two support members 12A and 12B that each support a microtiter plate 14A and 14B. Of course, various other embodiments can be used with two or more support members 12A and 12B. To coordinate their functionality, the support members 12A and 12B are connected to an external control device 36, such as a computer. In this application, the user may transfer fluid from wells 16 on one microtiter plate 14A or 14B to wells 16 on the other microtiter plate 14A or 14B. For example, the top center well 16 is illuminated on the top plate 14A of FIG. 8, while the top left well 16 is illuminated on the bottom plate 14B of FIG. 8. Accordingly, a technician may use a pipette to transfer fluid from the top center well 16 in the top microtiter plate 14A to the top left well 16 in the bottom microtiter plate 14B. In a manner similar to the process of FIG. 4, the light element 31 in the top tray may be turned off after fluid is withdrawn from it. In a corresponding manner, the light element 31 in the bottom tray may be turned off after fluid is added to it.

Some embodiments of the invention feature a “training mode” for generating computerized protocol scripts. A protocol script includes a series of instructions used by a computer to actuate visual indicia that guides a user to specific wells 16 for fluid removal and addition. The protocol script also may contain instructions for the computer to display instructive text to the user in real-time, and may automatically control the volume settings of a liquid dispensing instrument as well as other data from the dispenser that may be used to confirm a successful liquid dispensing or withdrawal event. For example, dispensers that use an air displacement mechanism could confirm a successful dispensing event by detecting internal air pressure changes. Protocol scripts may be saved as a computer file and shared with other users. For example, protocol scripts may be supplied on computer media, along with reagent kits, downloadable via the Internet, attached to emails, or archived on a website and referenced in scientific publications.

Among other things, various embodiments of the invention also may be used with multi-channel pipettes for parallel liquid handling operations throughout a row or column of a microplate (e.g., the Gilson Pipetman® Ultra Multichannel for Gilson, Inc., Middleton, Wis.). Handheld, electronically actuated pipettes (e.g., the Rainin EDP-Plus™, Oakland, Calif.) also should operate with the system 10. A greater degree of data logging for an experimental procedure should be obtained using an improved handheld electronic pipette having the capability of recording and transmitting its records of data regarding liquid transfer events; for example, the times of, and volume settings for, liquid-withdrawal and dispensing events. The pipette may also record and transmit pipette ejection events; such records may have usefulness, among others, in identifying likely well-to-well contaminations, such as by DNA, microbes or RNAse. This data could be transmitted via a built-in wireless transmitter, via a cable, or recorded in an on-board memory for later recovery. In this way, additional confirmation of correct experimental procedures may be obtained, and volume information may be automatically programmed into the protocol script in the “training” mode. Additionally, a protocol script may automatically adjust the volume settings of the pipette at different stages of the experiment based on the pattern of well penetration data. Recorded volume setting and pipettor actuation events are useful data, but further confirmation of correct liquid handling could be provided by a pipettor capable of sensing the actual presence of liquid in a pipette tip; such sensing may utilize among others measures, capacitance, optical properties, thermal properties, or acoustic properties of the pipette tip.

Various embodiments facilitate preparation of training scripts. For example, the system may be set to a programming mode that enables a user to manually perform a set of steps with regard to one or more microtiter plate 14s. The diodes 18 and detectors 20 detect and record the sequence of well accesses on some storage medium. A computer program thus uses these accesses as the basis for a program that leads a subsequent user through a procedure, such as that discussed above with regard to FIG. 4. Such a process should significantly simplify programming certain protocols.

Scripts may also be associated with a reagent kit. By using bar codes, 2-dimensional bar codes, RFID tags, RuBee (IEEE 1902.1), or other machine readable media (including media also readable by a human), a reagent kit may identify and incorporate a protocol script for use with the system 10. For example, scanning of a bar-code on a reagent kit box or instruction sheet may provide an identification code that will allow a computer to download an associated protocol from a magnetic media, optical media, or network. Alternately, the protocol may be stored on magnetic media such as a flash memory that is shipped with the reagent kit. Alternatively, the kit may include a piece of paper with an encoded script that may be read with a scanner associate with system 10. In yet another example, a system 10 may be programmed with a script for performing a particular reaction and shipped as part of the reagent kit for that reaction. A script identifier may also be affixed to one or more microtiter-plates that are shipped with the reagent kit.

Protocol scripts may incorporate features designed to increase user safety or environmental awareness. For example, warning messages may be displayed on a computer monitor when a step in a protocol is reached that utilizes a toxic or radioactive agent, or that generates a toxic, flammable or explosive product. Specific instructions may be displayed, or audibly announced to the user to advise the user to exercise caution, use proper safety techniques, or perform proper waste-disposal techniques.

Accordingly, various embodiments detect access to a given receptacle 14 of a fluid handling device, thus enabling a wide variety of additional applications. Moreover, it should be noted that discussion of manual use of the discussed embodiments is illustrative and not intended to limit the scope of all embodiments. Accordingly, the processes discussed above may be implemented with automated equipment, such as a robotic system that dispenses or otherwise handles fluids.

Various embodiments of the invention may be implemented at least in part in any conventional computer programming language. For example, some embodiments may be implemented in a procedural programming language (e.g., “C”), or in an object oriented programming language (e.g., “C++”). Other embodiments of the invention may be implemented as preprogrammed hardware elements (e.g., application specific integrated circuits, FPGAs, and digital signal processors), or other related components.

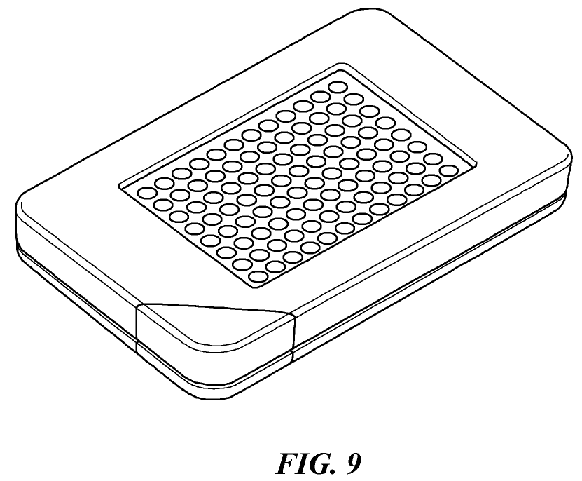

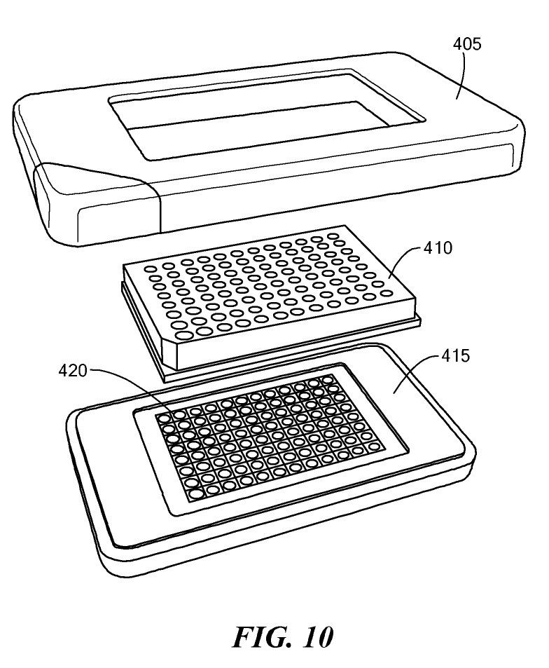

FIGS. 9-10 show a two-part support member 12. FIG. 9 shows the support member 12 in an assembled configuration and FIG. 10 shows the. A base unit 415 has a jig 420 to position a receptacle, here a microplate 410. A collar 405 is placed atop the microplate and contains a well penetration detection assembly. The jig 420 is shown with multiple holes for positioning the microplate 410, but omitting the holes would allow for more flexibility in the type of receptacles used. On the other hand, use of the multiple holes may serve to transfer heat to or from some or all of the microplate 410 if the base 415 is configured for temperature control. Use of a two-part support member allows the use of microplates of varying heights. The one-part support member 12 of FIG. 2 may require shimming in some instances.

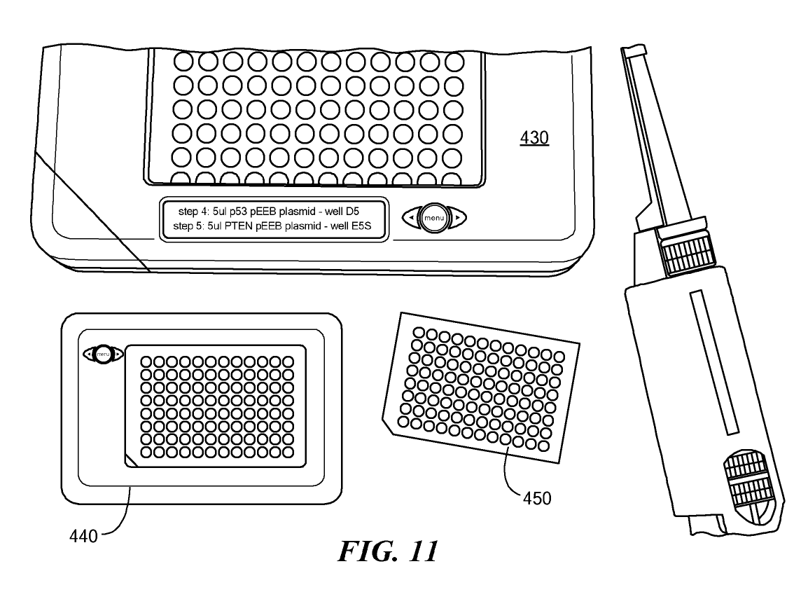

FIG. 11, shows a full format support member 430 and a smaller, sub-microplate format support member 440, with a complementarily sized microplate 450. The full format support member 430 has a multi-line digital display for communicating instructions and information to the experimentalist. The sub-microplate format support member 440 may be useful for performing smaller experiments, for holding source reagents, as an educational tool, etc. It may have all or only some of the functionality of the full sized device; thus, the various features described herein may be employed in both large and mini formats. The sub-microplate format support member 440 may, for example, have 96 wells on 192 or 384-well microplate format spacing (such spacing may optionally accord with Society for Biomolecular Screening [“SBS”] standards).

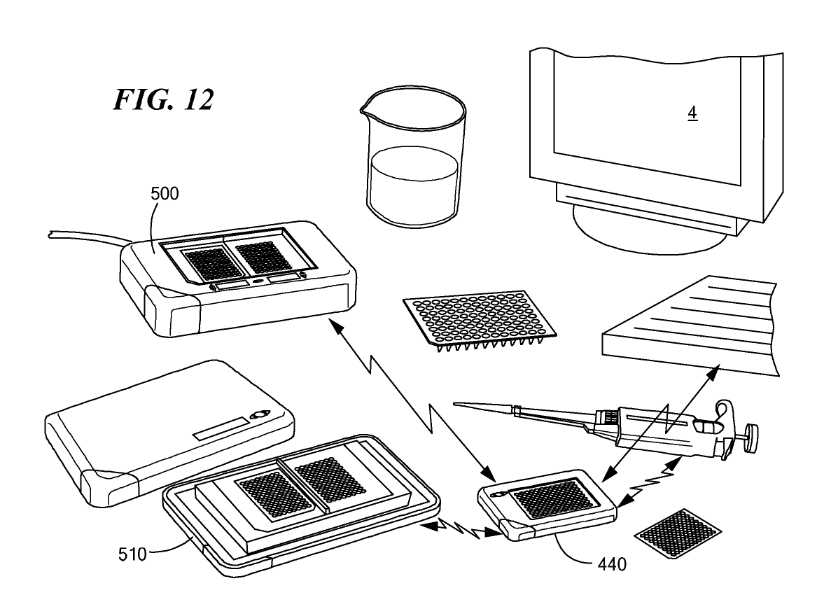

FIG. 12 shows a representation of an interactive benchtop system 10 containing multiple wirelessly networked laboratory devices. The system 10 has a computer, 4 a sub-microplate format support member 440, a temperature control companion device 500, a microplate fluorimeter companion device 510, and a pipettor configured for logging liquid-handling events and communicating the log wirelessly to the compute 4. Double-ended arrows represent wireless communication between the components. The communication is not limited to the arrows shown; all of the devices may potentially communicate with each other in a network. The network may also be linked in communication with remote devices, e.g., a LAN or WAN, including the internet. The microplate reader 510 is especially compact, in part because it has no control or data processing interface. Instead, it relies on associated system devices such as a compact data processing apparatus or wirelessly networked computer to provide data acquisition protocols and data storage and processing capabilities.

By creating such a laboratory network, experiments may be performed efficiently and reliably, and a data log created for analyzing or verifying the experimental procedure. For example, a reagent kit for nucleic acid sequence analysis using isothermal amplification may be performed (e.g., a Third Wave Invader assay). A reagent kit may come with a bar code pointer to an protocol stored on the internet. Scanning the barcode with the computer 4, will initiate download of the protocol. The system will instruct the experimentalist to insert various receptacles into the support and to mix the necessary reagents. The system will timestamp and log these various computer actions and sensed user actions (the system may also detect receptacle insertion events). At the appropriate step, the system will communicate with the heat block 500 to pre-heat the block and will instruct the experimentalist to transfer the receptacles to the heat block 500. The heat block 500 may detect the presence of the receptacles and initiate a timer, which may be located anywhere in the system. After an incubation time (e.g., 3 to 4 hours), the system will switch off the heat block power (or even cool to sub-ambient temperature) and summon the experimentalist with an alarm (e.g., voice, email, phone call, text message, etc) and instruct the experimentalist to transfer the receptacle 14 to the fluorimeter 510 for analysis. The system may use the well penetration detection log and/or temperature measurements from the heat block 500 to calculate reaction rates and may automatically generate a standard curve and compare the rates to the curve. The system may then generate a report for the experimentalist or supervisor.

Other companion device embodiments (not shown) are contemplated. For example: a small-format gel electrophoresis system, a gel imager, a scintillation counter, a spectrophotometer, a isoelectric focusing system, a microarray scanner or imager, and a robotic fluidic handling station (further described below).

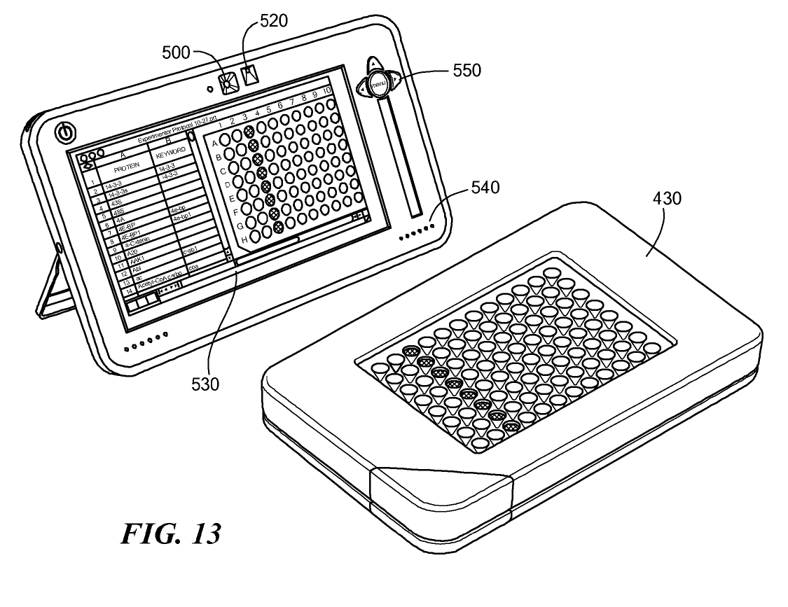

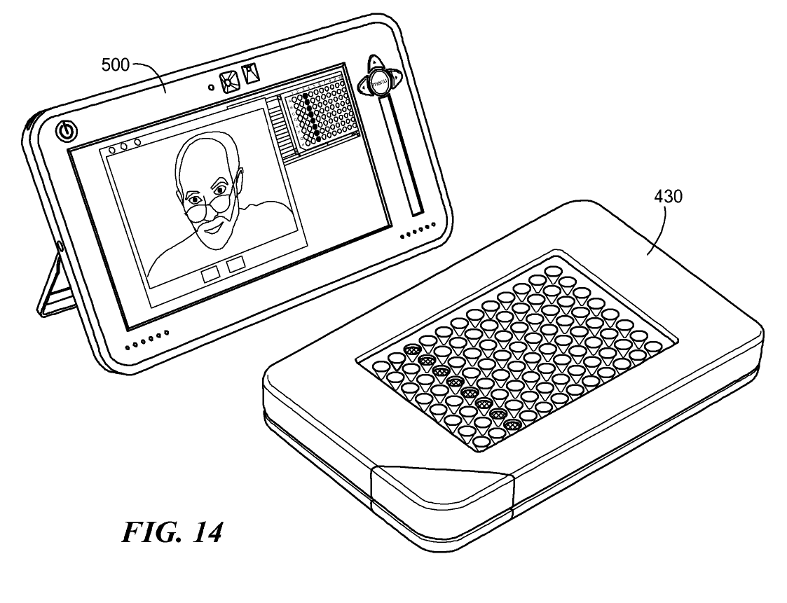

FIGS. 13 and 14 show a representation of a support 430 for a microplate 14 with a compact data processing apparatus computer 500 that is configured to be a lid for the support 430 when the support 430 is not in use. Alternately, the compact data processing apparatus 500 may be configured as a base to be positioned under the support 430. The compact data processing apparatus 500 and the support 430 may be clipably attached as with a friction fitting lips, clasps or other suitable mechanism. The compact data processing apparatus 500 may have a headset jack on its side (although it may use wireless headset technology such as Bluetooth). On the underside of the compact data processing apparatus, there is an on/off switch, an LCD display 530, stereo speakers 540, a microphone 520, a digital camera 510 (which may function as a bar-code reader), and scrolling and navigating controls 550 including fast-forward, rewind, play/pause, and volume controls. The display shows green indicia on a microplate map that may indicate, for example, that a user should add a reagent to column 3 of the 96-well plate held in the support 430. Inside the compact data processing apparatus, are housed the various electronic components to perform the various input/output, storage, logic and power functions required. The lid has a semi-rectangular support arm that is rotatably connected to the outside of the compact data processing apparatus; the arm allows the compact data processing apparatus to be attached to a shelf. The arm and/or shelf may include additional support structures such as magnets or clamps to more firmly position and support the compact data processing apparatus 500.

In an embodiment, the camera may be mounted on the front or back of the compact data processing apparatus 500. The camera may be used to take photographs of wells in the microplate, and so may be fitted with appropriate magnifying optics. The camera may, with an appropriate strand be used as a detector for colorometric, fluorometric or luminescence detection modalities. Illumination may be provided be LEDS in the base member, by the LCD display, or by other sources. In a related embodiment, the camera may be configured to be on the side of the compact data processing apparatus 500 opposite the display so that images of the wells may be displayed on the display prior to activating image recordal.

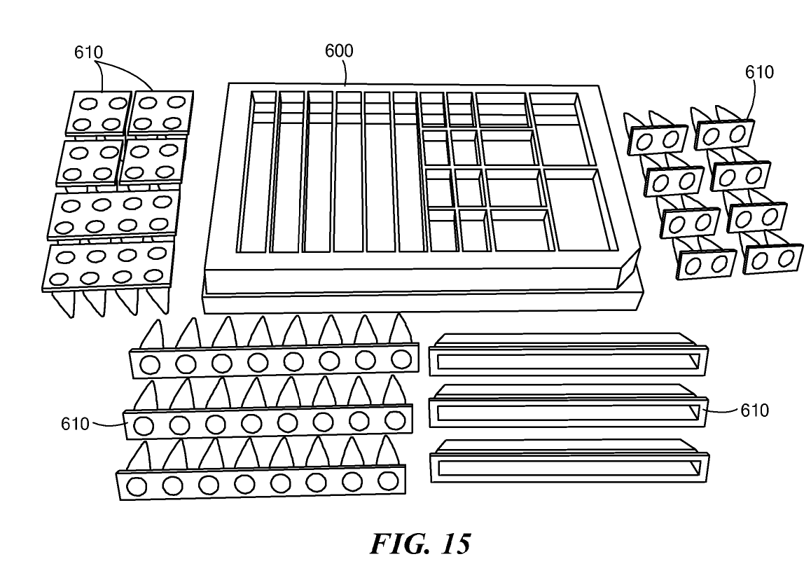

FIGS. 15-19 illustrate embodiments that utilize a modular receptacle system. FIG. 15 shows a disassembled mix and match system. The system includes a plastic frame 600 having microplate dimensions (e.g., SBS dimensions) adapted to hold multiple types of insertable receptacles 14, hereinafter referred to as “inserts 610.” Each insert 610 may have one or more wells 16 in various geometries, including a trough geometry adapted for the supply identical reagent to multiple tips of a multichannel pipettor. Although many configurations are possible, the frame 600 shown is adapted to hold troughs and well-strips; the well-strips shown here are spaced according to a 96-well format. Some of the strip inserts 610 shown may also held together by frangible tabs, which may be torn by an operator to separate the multiple strip inserts 610. The inserts 610 may be filled with reagents prior to delivery to the operator. The inserts may be preassembled into a kit, and the kit may optionally be pre-assembled in a frame prior to delivery to the operator. Alternately, the operator may assemble various inserts 610 into the frame 600 to arrive at a completed kit. In addition to reagents, a kit may include various devices (i.e., device inserts 610) that are attachable to the frame 600, either using slots as shown in FIG. 4, or using features for attaching to the frame periphery. Examples of devices that the frame 600 may accept include pipette tips, a magnet for bead separation, a microscope slide, a cell culture dish or chamber, a small electrophoresis gel, hybridization-based biochip, a pump for a microfluidic device, a power supply, a miniaturized capillary chromatography system or other microfluidic device, a vibrational mixer (e.g., piezoelectric or motorized buzzer), rocker, among others may be insertable into the frame 610. The frame 600 may be wired to provide power or communication to the device insert upon insertion. Alternately, the device insert may carry its own power supply (e.g., a miniature fuel cell or a battery, including a rechargeable, disposable or thin-film battery), if needed, and may use wireless communication. Optionally, a piezoelectric vibrator is built into the insert. The kits may also include electronic instructions; in an embodiment, the frame includes a holder for a computer chip, memory card or other such device that contains a protocol for uploading to a compact data processing apparatus or other networked computer.

Inserts 610 may be configured to be gas resistant by coating a plastic with a layer of metal or glass, or by constructing them from metal or glass. This will facilitate performance of air sensitive reactions such as polyacrylamide polymerization.

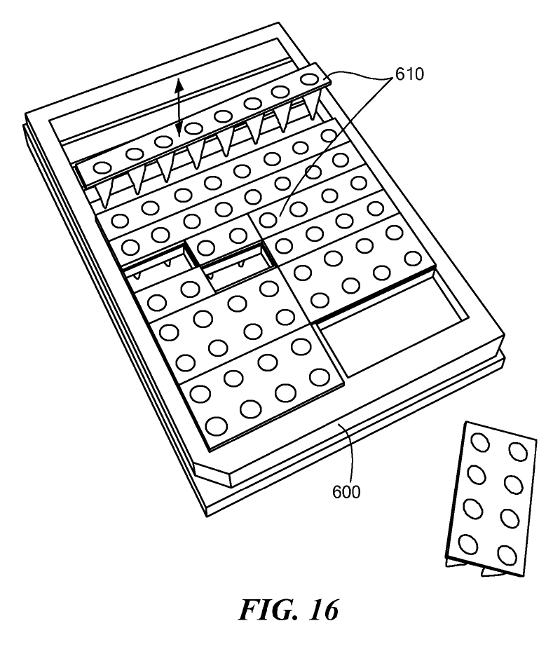

FIG. 16 shows a user introducing inserts 610 into the frame 600. Device and reagent inserts 610 may be secured via mating features, such as a friction-fit, pins, or tabs, or, alternately, using an adhesive. The inserts may be difficult to remove from the frame thereafter. Accordingly, in an embodiment of the invention, features are included for facile removal of inserts 610 from a frame 600. For example, a hole in the portion of the insert facing the operator and bordering the functional region of the insert (well, trough, device, etc.) allows the operator to remove the insert using a plastic pipette tip (e.g., a standard yellow 200 microliter tip). Alternate embodiments are also contemplated, e.g. a loop on the surface of the insert.

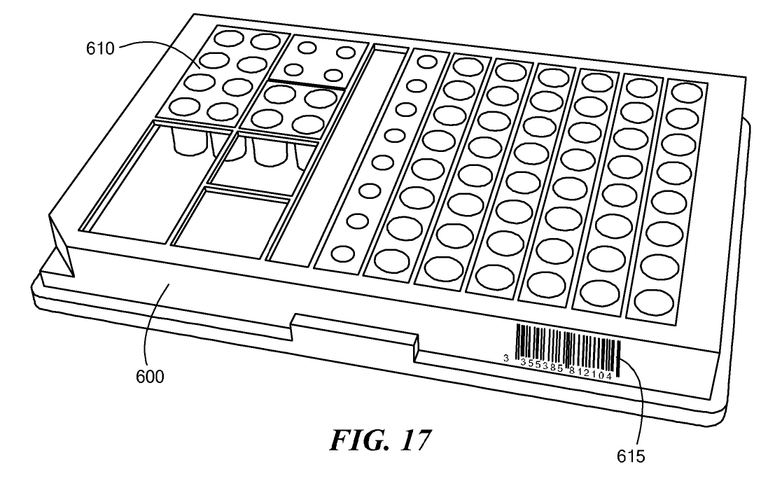

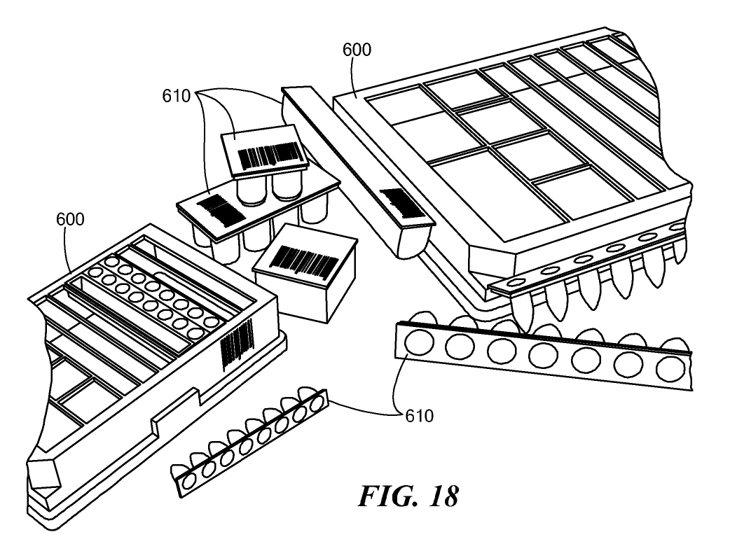

FIG. 17 shows a frame 600 with a bar-code 615 and transparent inserts 610. The wells 16 of the inserts 610 are shown in both higher and lower volume formats. FIG. 18 shows frames 600 in microplate and sub-microplate format, for use with a sub-microplate format support (item 440 of FIG. 12).

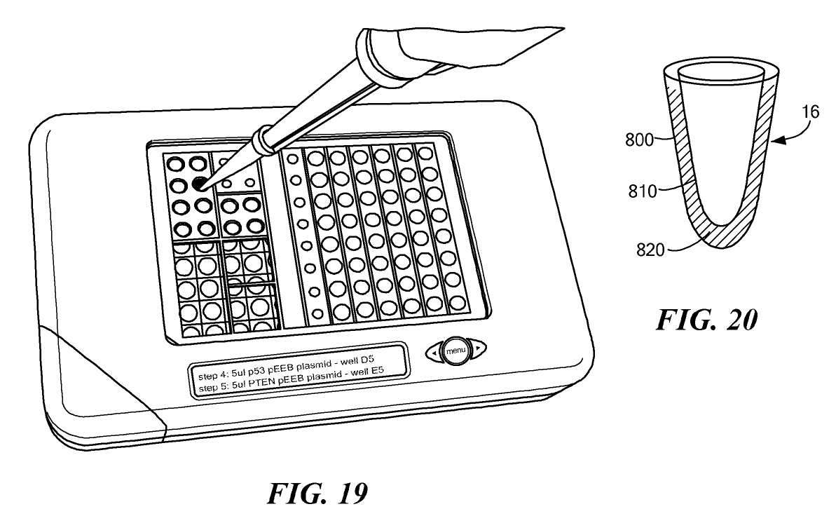

FIG. 18 shows frames 600 in microplate and sub-microplate format, along with sealed and unsealed insert receptacles. Reagent inserts 610 may be covered in one or more layers of peelable or pierceable film. If sterility is required, the film may be chemically sanitized by the operator prior to use, e.g., swabbing with 70% ethanol. Alternately, the insert is provided with a double film; the upper layer of film is peelable to expose an underlying sterile layer that is then pierced using a needle or pipette tip. Note that the reagent inserts 610 may also be provided in an empty condition to provide a clean sterile, receptacle 14 for the user to run a reaction in. FIG. 19 shows a frame 600 and receptacles 610 in a support 12 during use.

Reagents may be packaged in the inserts 610 by a reagent or kit manufacturer. Since different reagents may require different shipping storage conditions (e.g., room temperature, 4° C., or on dry ice), different reagents needed to complete an assay or reaction kit may be assembled by the operator. For convenience, the reagents may be commonly colored or otherwise coded. Machine-readable tags on the inserts will allow detection by a compact data processing apparatus or networked RFID tag or bar-code RuBee, or other reader. The tags may also encode information related to the reagents including reagent identity, lot number, production date, expiry date, and storage conditions, concentration, or specific activity. The system may alert the operator prior to initiation of the protocol if an error has been made in selecting and assembling the inserts 610. Additionally, the inserts 610 may include indicia of improper storage conditions; for example, an indicator that changes color upon exposure to unduly high temperatures.

Reagent or device inserts 610 with machine-readable tags may also be used in connection with an inventory management system. A compact data processing apparatus or other networked laboratory device will detect the presence of the insert and correspondingly alter an inventory counter. If the counter drops below a given threshold value, the system will either notify a human or automatically order additional inserts. Optionally, the system will communicate with the supplier to obtain estimated delivery times. The system may use these delivery time estimates by notifying a user of a likely component shortage or by adjusting the threshold value accordingly. For example, the device may increase the threshold value in response to an elevated lag-time. The system may also decide when to initiate reorder or reorder notification based on the consumption rate of a given type of insert. Alternately, insert codes may be manually entered into the computer.

Inserts 610 may also be configured to have a high heat capacity so as to maintain a given temperature for a period. The high heat capacity may be used to keep a well cold upon removal from frozen storage. FIG. 20 shows a well 16 of an insert 610 that has a multi-layer construction. The well 16 has an outer plastic layer 820, an inner plastic layer 810, and a high heat capacity material 830 therebetween. The high heat capacity material 830 may be a liquid such as water, salt solution, glycerol, DMSO, or mixtures thereof, may be a gel material such as acrylamide or agarose, or may be a solid such as particulate metal. Using a phase-changing material such as a liquid or gel may have the advantageous effect of keeping the material at a temperature around the melting temperature for an extended period and therefore gives a greater degree of temperature control. For example, the phase change material may be chosen with a melting point around 4° C., which is optimal for keeping many biochemical reagents in a liquid but stable condition. One problem that may be encountered in constructing the multilayer insert 610 is that if the inner and outer layers are sealed with a phase changing material between and the material is frozen, and the material expands upon freezing (e.g., water), the expansion will cause distortion of the insert 610. To overcome this problem, a material that contacts upon freezing may be used. Alternately, the layers may be assembled with the material in a frozen state and then cooled to create a vacuum between the inner and outer layers. For example, one may place frozen gel in the outer layer, insert the inner layer and fuse by welding. Alternately, a liquid may be introduced into the outer layer, frozen, and the inner layer added and the device welded together while the liquid is still in a frozen state.

In an embodiment of the invention, an outside surface of one or more reagent inserts 610 is sprayed with a ceramic resistive material that allows it to be heated by means of application of an electric current (see, e.g., U.S. Pat. Nos. 6,924468; 6,919,543; 7,123,825; and 7,041,944 and www.thermoceramix.com). By using a thin layer of resistive material, the insert will also cool very quickly. Such an arrangement may be useful for performing high and/or low temperature incubation or thermal cycling steps. Thermistors may also be built-into the inserts to provide real time monitoring and feedback control. A thin film battery may also be incorporated into an insert 610 to supply power for heating, electrophoreses, electroporation, etc.

As mentioned above, frames 600 may be loaded into the support 12 for use with a system's guiding or other features. The support 12 may sense the presence of the frame 600 and/or individual inserts 610; such sensing may be wireless (e.g., using RuBee). Protocols may be downloaded (e.g., from a networked computer or the internet) to the compact data processing apparatus based on a machine-readable identifier associated with a frame 600, insert 610, or collection of inserts 610. After completion of the protocol, data may be uploaded to a computer 4 in order to generate a report.

In an embodiment, an operator uses the support 12 as a sample collection device. For example, the support 12 can receive bar-coded blood samples contained in bar-coded evactuated collection tubes (e.g., Vacutainers®), buccal swabs or forensic samples. The compact data processing apparatus may record various parameters including collecting operator identity (which may be biometrically verified by, for example, an onboard fingerprint reader), other chain-of-ownership data, patient or victim data, collection time, collection location, etc. Additionally, peripheral tools for sample collection may be attached to the support 12, or to a drop-in disposable component (e.g., a frame 600). Examples of peripheral tools include swabs or scrapers for obtaining buccal or other cell samples, and phlebotomy sets. Elongate collection devices such as swabs will tend to protrude awkwardly from a corresponding holder frame in the compact data processing apparatus. To avoid this problem, shorter swabs (or other collection device) may be used; a peripheral tool, which may be attached to the support 12, may grab the shorter swabs; the tool attaches to a swab to allow collection, and detaches after the swab is deposited in the holder frame.

In an embodiment of the invention, a liquid handling robot is adapted to execute a protocol script associated with a reagent kit and may work with one or more components of a system 10 such as a support 12, a frame 600, and a companion device 8. In an illustrative example using a robot, a compact data processing apparatus is loaded with a frame 600 and multiple receptacles 610 of a reagent kit and at least one test sample for analysis. The support 12 is positioned into the robot using a positioning jig. The robot may sense the presence of particular reagent inserts 610, empty well inserts 610, and device inserts 610 in a frame 600, or this information may be downloaded to the robot from a support 12 or associated system component. The robot obtains instructions regarding the sequence and timing of liquid handling steps (i.e., a protocol script 2). The robot then automatically pierces reagent inserts, withdraws reagents and dispenses them into a sample-containing well, incubates for a time period, and then adds a stop reagent. The reaction well is then monitored for end-point absorbance using a fiber-optic based spectrophotometer.

In an embodiment, a system 10 is adapted for educational use. The support 12 may be a sub-microplate format support 440. The compact data processing apparatus 500 may utilize a video display (e.g. LCD, or virtual reality-type goggles) and onboard digital media or streaming circuitry to present a lesson to a student. The display may also be located on a student's cell phone, mp3 player, personal digital assistant, portable game player, or other such portable device. Examples of lesson media include hypertext, video, and interactive video. For example, the lesson may include molecular simulations, protocol instructions, and/or quizzes (with interactivity via a touch-screen or other input device). Answering a quiz may be required to allow the student to advance the protocol and receive the next instruction. The display may also show time warnings, or other alerts.

User performance may be tracked and rewarded with points, which may be redeemable for teacher accolades, valuable prizes or both. Student progress and device status may be periodically automatically uploaded to the teacher's computer. Different students in a class may receive different experiments to avoid cheating, or may receive different test samples. Data collected by each student may be automatically compiled over the wireless classroom network to allow for statistical analysis of the results by the students or class.

For maximum convenience, the system may include, as inserts, all the components necessary for a classroom laboratory experiment. The components may include reagents (including water) a simple liquid transfer tool (such as a pin, loop, or bulb-type pipette). The compact data processing apparatus may be in wireless communication with a system computer (e.g., on the teacher's desk, or a hand-held computer) and/or networked with other student's devices, and shared companion devices.

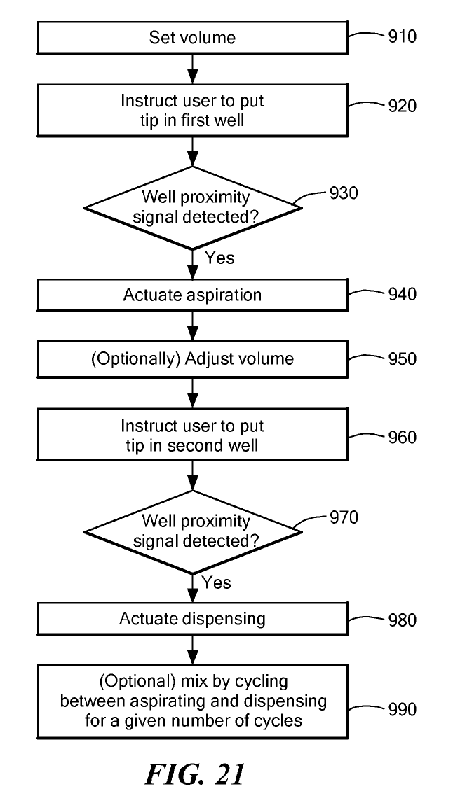

FIG. 21 is a flow chart showing an embodiment that includes semi-automated execution of a liquid handling procedure using a protocol script in conjunction with a support 12 and an instrumented pipette companion device. Prior to beginning the diagrammed portion of the procedure, the user prepares a reagent kit by inserting a frame 600 and receptacles 14 into a support 12 configured for pipette tip-well proximity sensing. The bar code on the frame serves as a pointer to the appropriate protocol that is downloaded from a computer network. The receptacles having reagents constituting a kit along with empty wells for mixing reagents and samples and are covered in a peelable and a puncturable layer. The user peels off the peelable layers and disposes of them (alternately, bar codes on the top layer can be scanned to provide information about the receptacle contents). The support uses RuBee detection to identify the receptacles, checks their expiration dates against an internet database maintained by the supplier, records their relative positions and/or orientations and uses this information in conjunction with the protocol script to direct the user to the appropriate wells at the appropriate times during execution of the procedure. The support than instructs the user via flashing indicia and instructions on a two-line display (e.g., an LCD or organic-LED display) to load the samples into the appropriate wells.

The reaction procedure is now ready to begin. An associated computer 4 initiates a wireless signal to the instrumented pipettor to adjust its dispensing volume to the appropriate setting (step 910). The user is then instructed to put a pipette tip on the pipettor and to withdraw fluid from a given well (or multiple wells if a multichannel pipettor is used) at step 920. The system then awaits detection of well penetration with the tip for a threshold time of 1 second (step 930), and initiates withdrawal of fluid from the well (step 940). Optionally the computer may alter the volume setting of the pipettor (step 950); this may be occur, for example, if multiple dispensing operations are to occur using the same withdrawn volume. The computer then instructs the user to move the pipette tip to a new well location (step 960) and the system awaits detection of well penetration (step 970). Upon detection of penetration that is maintained beyond a threshold time, the system actuates dispensing of the fluid in the pipette tip (step 980) by sending a wireless signal to the pipettor. Optionally, the system actuates the pipettor to cyclically withdraw and aspirate to effectuate mixing of the dispensed fluid with the well contents. Each of the above steps are time-stamped and logged by the system.

### Example A

LED's could be timed to indicate exactly when someone should dispense into or pull sample out of a well. For example, a flashing light could signal that an incubation time is over. The system could light up (at very low intensity) all the wells that will be used during each major step in the experiment. Users could then see the “road-map” of next steps. There could be a gradient of intensity, such that the wells closest to the next pipetting step are slightly higher intensity than the wells further away in the experiment. These intensities would be much lower than the active wells, but enough to see.

### Example B

A compact data processing apparatus may be sized to be comparable to the support. For example, the length and width or the compact data processing apparatus may be less than or equal to twice the length and width of the support, respectively. The compact data processing apparatus and support may mate; e.g. with clips, a friction fitting lip, or other mechanism. The compact data processing apparatus may be removable from the support and may have various useful features, for example, a display screen, a touch sensitive screen, speakers, web cam, microphone, hard drive, RFID reader, bar code scanner (alternately, the camera could also function to read bar-codes), detachable micro-mouse, and jacks for ear plugs. It can be wireless or connected and have a rechargeable battery. If the portable computer is battery powered, it may be charged with a separate charging station, this function may be also be accomplished by the support. The compact data processing apparatus should also have a mini flip-out stand so that it can be positioned at various angles on the benchtop. The compact data processing apparatus may be capable of storing large amounts of information, protocols, data, music or other digital media. A docking station or plug-in charger could be included.

The compact data processing apparatus could be used as an audio-visual experimental guide and/or as a communication tool (e.g., using voice over IP, or streaming digital video protocols). It can also have a stand or clamp that positions such accessories at a convenient height for visual and verbal communication at the bench or desk top and allow for videoconferencing with a favorable viewing angle. The compact data processing apparatus could be lifted up into a viewing plane or held within a viewing plane that is conducive to someone standing or sitting at the benchtop. This could be, without limitation, a mini podium stand (for example, a clear bent acrylic stand similar to a pipette holder) or a bookshelf style adaptor that mounts onto a shelf and extends at a downward angle. One may then place the compact data processing apparatus in front of them at a convenient angle for experimentation and communication. A second camera may be included for use as a bar-code reader, or to make a photographic or video record of experimental steps.

The compact data processing apparatus may also incorporate a digital music player (e.g. mp3 player), AM/FM radio, global positing system, or other consumer electronics functions.

### Example C

The system may include the ability to easily communicate to a user whether a given step in a protocol requires liquid dispensing or withdrawal from a given well. For example, different colors or other optical signals could be illuminated to distinguish transfer vs. withdrawal. For example, a flashing light could indicate the need for withdrawal of fluid and a solid light could indicate the need for dispensing. The screen could also differentiate each step easily. An icon could be used (a pipette tip with a slashed circle over it) plus a pleasant tone to indicate when tips need to be changed.

### Example D

The computer's software could optimally organize the experiment on the plate. The computer could keep track of which wells were previously used and so conserve wells in microplates by allowing users to utilize these plates without a high risk of accidentally using contaminated or used wells. This is especially useful if the wells have been provided to the user with expensive reagents, or if the plates are used to store samples and space is scarce.

### Example E

The system may operate as a laboratory notebook. Experimental details and results may be entered by a user. A time-stamp or other authentication data may be included and may be co-encrypted with the experimental details. An associated network may automatically backup the data. The information may be placed in a tamper-resistant, read-only format to help ensure the authenticity of the information. A digital signature may be included; for example, an authentication string generated upon entry of a password or with the use of a keycard. The authentication process may also utilize biometric data such as a fingerprint, retinal scan, facial recognition, or voice recognition. The digital signature of a witness may also be collected. Video and audio recordings made by the system may be included in the notebook.

### Example F

A computer associated with the system may print one or more tags for inclusion on page of a lab notebook. The tag may include an identifier may have an adhesive and peelable backing so that it may be easily attached to a notebook page. The identifier may be a pointer to one or more database records that may include data such as the experimental protocol script and an associated data file that may aid in determining the validity of the experiment in terms of the proper liquid handling steps. The records may also contain data such as the timing liquid handling steps and ambient temperature or humidity measurements that may be useful in the analysis of the data. The tag may also directly contain a record of such data. The tag preferably includes machine-readable information (e.g., bar-code, 2-dimensional bar-code, or RFID tag).

### Example G

The system may include a printer for printing labels for reagents, tubes and the like. The labels may be color-coded and may have text, machine readable information (e.g., bar codes) or a combination thereof. As an alternative to bar codes, the labels may be programmable wireless tags; computer transfers information to the tags, the tags are affixed to reagents-holding vessels, and the information is retrieved during an experiment. The labels may serve varying purposes, but generally are an aid to a user, or used for tracking quality. For example, the labels may include the identity of a reagent, lot number, and information related to an expiration date or time (e.g., for labile reagents such as unstable aqueous mixtures of TEMED or APS) commonly used in electrophoresis gel casting. A bar code scanner can be used before or during an experiment to record information related to the reagents being used. If the wrong reagent or an expired reagent is being used, the user may be alerted and a note may be included in an experimental report. A detachable bar-code scanner may be used to allow scanning of large or heavy items (e.g., a drum of acetonitrile). A bar code scanner (or RFID reader, etc) may also be used to record the identity of a piece of analytical or preparative equipment and the time at which it was used.

### Example H

An intelligent benchtop may be used to interact with various experimental tools and supplies. Such an intelligent benchtop typically has sensors for detecting the layout various components that will be used in an experiment. The sensors may be disposed throughout a mat, built into a solid surface or be disposed around a working surface and sense pressure or communicate wirelessly with the components. Examples of components might include, a support member 12, a compact data processing apparatus, a microplate, a rack of microfuge tubes, a 15 or 50 ml conical centrifuge tube, a box of gloves, a pipettor, a box of pipette tips, etc. The system may alert a user to the need for additional items on the benchtop, or the presence of an inappropriate or defective item such as an expired or unsafe reagent. The benchtop may include visual indicia such as lights, LEDS or displays to communicate the position of a component needed in a particular step of an experimental protocol.

### Example I

A user may be provided with a ring, watch, bracelet or other wearable item that may aid the system in identifying the position of a user's hand relative to the position of various reagents or components, or relative to fixed components of an intelligent benchtop. In this way, evidence may be collected to help determine that the proper reagents, instruments, or other components may be used in a procedure, and warn a user if they are using incorrect components.

### Example J

For some procedures, a dangerous or restricted item or substance may be used. For example, some procedures require the use of highly toxic or caustic substances, drugs of abuse, radioactive materials, precious materials, or dangerous pathogens. In such instances, it may be desirable to have automated security systems that are automatically activated by the system. For example, a high-security freezer may unlocked in response to a command from the system; the freezer may also require proximity of a bracelet as described in Example I and a biometric impression. In this way, access to the material can be associated with the performance of a particular experiment and access can be denied at times when experiments are not authorized. Additionally, the system may track the performance of safety-related steps. For example, sensors associated with a box of laboratory gloves may provide information related to times at which the entrance to the box was penetrated by the hand of a user (e.g., through proximity sensors or detection of a bracelet with an RFID tag). One or more steps in a protocol may be automatically forbidden or discouraged if the proper safety steps have not been detected. The presence of authorized safety glasses may be detected; for example by the presence of an RFID tag affixed or embedded in the safety glasses. The presence of an authorized supervisor may also be detected by the system and required to proceed to sensitive portions of the experiment. The system may communicate a detected emergency or unauthorized access of materials to a security agency or alarm system.

### Example K

An application specific system or system component (e.g. support member 12, or compact data processing apparatus) may be provided to a user. The system component may be designed to work only with a particular assay or group of assays. The system may be activated by identifiers associated with a reagent kit or particular reagents or experimental tools. The component may be packaged and shipped with a reagent or reagent kit.

### Example L

The system may track penetration, not to closed-end wells of a microplate, but to various other ports. For example, sensors may detect the addition of samples to a capillary electrophoresis system, mass spectrophotometer, lab-on-a-chip, microarray, collection vial, etc. The sensors may also detect addition of fractions from a fraction collector to wells of a microplate. The support member may also hold filtration microplate assemblies and provide vacuum or pressure to drive liquid samples through the filters. The support member may include permanent magnets or switchable electromagnets to separate magnetic beads from solutions in the receptacles.

### Example M

Particular system configurations may be useful in the collection of samples in the field. In such a configuration, the system is preferably lightweight and compact. The system may have a support member for holding an array of receptacles such as tubes or wells into which solid (e.g. fibers, hairs, swabs, toothpicks and the like) or liquid samples are placed. Penetration of the receptacles is automatically recorded along with parameters such as the output of an onboard clock. Additional parameters that can be automatically recorded include user-entered text or digitized audio notes, temperature, humidity, and global positioning readings, or other data supplied by companion devices. GPS readings are particularly advantageous for the verification of forensic evidence in criminal or civil investigations.

### Example N

The support member 12 and other components may be sterilizable; for example by the use of bleach, or by autoclaving. A sterilizable liner may be included in the support member 12, which is resistant to aggressive sterilization procedures such as bleaching or autoclaving. The support member 12, may also advantageously be autoclavable, waterproof, withstand low or elevated temperature, be chemically resistant, or combinations thereof. The system and support member may be ruggedized to resist shock and be resistant to electromagnetic interference (EMI-RFI).

### Example O

The support member may vibrate a receptacle 14 to achieve a mixing step in a protocol script, or may be rugged enough to be vibrated or vortexed (held against a vortexing mixer). Vibration may be detected and recorded. Mixing may also be accomplished by repeated actuation of a pipettor to withdraw and dispense fluid; in this case, the system may communicate with an instrumented pipettor to verify that this mixing procedure steps was performed.

### Example P

In some embodiments, various sensors are integral to the receptacles (e.g. microplate). For example, a microplate may include conductivity sensors for measuring pH over time and the pH information may be transferred to the support member 12. The support member 12 may have a built in power supply for performing electrophoresis in the receptacles, or to power an external electrophoresis system.

### Example Q

In embodiments, the system tracks events associated with potential spills or cross contamination. For example, an accelerometer and/or level sensor may detect when a support member 12 is bumped or jarred so that liquid may splash from one well 16 of a receptacle 14 to another. The system may also track the ejection of pipette tips into a receptacle 14 equipped with a sensor to detect the ejection step; alternately, an instrumented pipettor may communicate a tip ejection step to the system.

### Example R

The system may allow a user to enter notes during or after the performance of the experiment. These notes may be in the form of audio recordings, which may be associated with a start time, and end time, and the particular step in a procedure script. The addition of an audio note may be initiated by an audio command such as “add note.” For example, a user could leave a note that says “add note, wrong reagent added to well A6”, save as audio and converted text file”. The existence of such notes could be included in an experimental report.

### Example S

A scanner or digital camera associated with the system may be used to scan materials other than bar codes. For example, the scanner or camera may record pages of a notebook and the system may use optical character recognition to convert images to text. The camera may be used to record experimental progress at various stages of an experiment and the photographs or digital movie files may be associated with particular protocols or notebook entries (paper or electronic). Data may be extracted from scanned or photographed objects; for example, the system may act as a densitometer to recover intensity values from a radiogram.

### Example T

In some embodiments, data from peripheral devices may be used to automatically customize a user protocol, using conditional rules encoded in the protocol script. For example, a microparticle manufacturer sells a kit for conjugation of antibodies to their microparticles. The kit includes premeasured antibody activation reagents stored in containers that are compatible with a modular container system similar to those discussed earlier (e.g., see FIGS. 15-18), premeasured aliquots of microparticles, protein detection reagents for measuring antibody concentration, and a USB flash memory device carrying a protocol script. After loading the protocol script into the compact data processing apparatus, the script instructs the user to dispense the protein detection reagents into the appropriate receptables, and guides the user through a manual dilution of the antibody sample to be used for the conjugation. After mixing the diluted antibody samples with the prepackaged detection reagents, the protocol script cues the user to transfer the plate frame, with associated receptacles, to a networked calorimetric plate reader. The reader transmits the absorbance data back to the compact data processing apparatus, and the compact data processing apparatus uses the data and with a calculation subroutine from the protocol script to calculate the concentration of the user's antibody preparation. The protocol script next calculates the desired dilution factor and directs the user to dilute his antibody to the proper concentration and amount for use with the kit reagents.

It is within the scope of the invention to use Examples in fields of endeavors including basic research, clinical diagnostics, education and training, forensics, sample collection, veterinary diagnostics, cell and tissue archiving, food testing, environmental testing, drug testing, cell culture, etc. Alternate Examples include using stackable inserts 610.

In alternative embodiments, the disclosed methods for guiding, communicating, and detecting use of fluid handling instrumentation may be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or analog communications lines) or a medium implemented with wireless techniques (e.g., microwave, infrared or other transmission techniques). The series of computer instructions embodies all or part of the functionality previously described herein with respect to the system. Those skilled in the art should appreciate that such computer instructions can be written in a number of programming languages for use with many computer architectures or operating systems.

Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). Of course, some Examples of the invention may be implemented as a combination of both software (e.g., a computer program product) and hardware. Still other Examples of the invention are implemented as entirely hardware, or entirely software (e.g., a computer program product).

The described Examples of the invention are intended to be merely exemplary and numerous variations and modifications will be apparent to those skilled in the art. All such variations and modifications are intended to be within the scope of the present invention.

What is claimed is:

1. A system for guiding reagent manipulations, the system comprising:
a support adapted to hold a plurality of sample receptacles;
a computer adapted to execute a protocol script so as to present a first instruction to an experimentalist, the computer sized to be comparable to the support;
a protocol progress sensor adapted to cause the computer to advance the script so as to present a second instruction in response to a given degree of protocol progress.

2. A system according to claim 1, wherein the computer reversibly attaches to the support.

3. A system according to claim 1, wherein the computer and the support are clipably attachable.

4. A system according to claim 1, wherein the computer is one of a base or a lid to the support.

5. A system for storing and assembling a plurality of reagents, the system comprising:
a frame having a plurality of differently sized or shaped cavities, the frame having a microplate footprint;
a plurality of inserts sized to be inserted into the plurality of cavities, at least one insert comprising at least one fluid receiving member.

6. A system according to claim 5, wherein an insert is sealed with a, puncturable film.

7. A system according to claim 5, wherein an insert is sealed with a peelable film.

8. A system according to claim 5, wherein an insert is sealed with a puncturable film and a peelable film, the peelable film adhered atop the puncturable film.

9. A system according to claim 5, wherein at least one fluid receiving member has an outer layer, an inner layer, and a high heat capacity material therebetween.

10. A system according to claim 9, wherein the high heat capacity material is one of a gel and a liquid.

11. A system according to claim 9, wherein the high heat capacity material is a liquid.

12. A system according to claim 9, wherein the high heat capacity material changes phase at a predetermined temperature.

13. A system according to claim 12, wherein the predetermined temperature is about 4° C.

14. A system according to claim 9, wherein the space between the outer layer and the inner layer is at a sub-atmospheric pressure.

15. A system according to claim 9, wherein at least one insert comprises an analytical device.

16. A system according to claim 5, further comprising one of a protocol and a pointer to a protocol, the protocol including a series of instructions associated with reagent or sample manipulation steps of a procedure to be implemented by an experimentalist, the protocol being in a format readable by a computerized apparatus, the apparatus adapted to accept input related to a previously performed step and to use the input to select an instruction for the user as to how to perform a future step.

17. A system according to claim 5, wherein at least one insert includes machine readable data related to the contents of the fluid receiving member therein.

18. A system according to claim 17, wherein the data relates to one of reagent identity, lot number, production date, expiry date, and storage conditions, concentration, and specific activity.

19. A system according to claim 5, wherein at least one insert comprises a feature for facile removal from the frame, the feature being selected from the group consisting of a hole, a tab, a pin, a lip or a flap.

20. A system for guiding reagent manipulations, the system comprising:
a support adapted to hold a frame having a plurality of differently sized or shaped cavities for holding a plurality of inserts sized to be inserted into the plurality of cavities;
a computer adapted to execute a protocol script so as to present a first instruction to an experimentalist;
a protocol progress sensor for causing the computer to advance the script so as to present a second instruction in response to a given degree of protocol progress,
where the support includes a reader for receiving machine-readable data physically associated with the receptacle.

21. A system according to claim 20 where the reader comprises one of a wired connector and an antenna for receiving a wireless signal.

22. A method for manufacturing a kit, the method comprising:
associating a reagent with a protocol script;
packaging the reagent in an insert adapted to mate with a frame having a complementary cavity;
providing the reagent and the script to an experimentalist.

23. A method according to claim 22, further comprising, providing the frame to the experimentalist.

24. A method for performing a reaction, the method comprising:
a) providing a frame having cavities;
b) inserting the frame into a support having at least one sensor adapted to provide information related to the degree of completion of a sample manipulation procedure;
c) inserting an insert into the frame, the insert having a receptacle;
d) receiving an instruction from a computer running a protocol script;
e) removing a liquid from the receptacle; and
f) transferring the liquid to a second receptacle.

25. A method according to claim 24, wherein step c) is performed prior to step b).

26. A system for use in semi-automated liquid transfer procedures, the system comprising:
a support adapted to hold a plurality of sample receptacles;
a computer adapted to execute a protocol script so as to present a first instruction to an experimentalist;
a protocol progress sensor adapted to cause the computer to advance the script so as to present a second instruction in response to a given degree of protocol progress;
an instrumented pipettor having programmably adjustable volume settings and a wireless communication assembly,
wherein the pipettor transfers records of liquid handling events to the computer.

27. A system according to claim 26, wherein the protocol progress sensor sends a signal to the pipettor in response to detecting a well penetration event.

28. A system according to claim 27, wherein the protocol progress sensor detects an occlusion of a light source.

29. A system for use in semi-automated liquid transfer procedures, the system comprising:
a support adapted to hold a plurality of sample receptacles;
a computer adapted to execute a protocol script so as to present a first instruction to an experimentalist;
a protocol progress sensor adapted to cause the computer to advance the script so as to present a second instruction in response to a given degree of protocol progress;
an instrumented pipettor having programmably adjustable volume settings and a wireless communication assembly,
wherein the protocol progress sensor actuates the pipettor to perform an action selected from the group consisting of adjusting a pipettor volume setting, actuating liquid withdrawal, and actuating liquid dispensing.

30. A system according to claim 29, wherein the protocol progress sensor sends a signal to the pipettor in response to detecting a well penetration event.

31. A system according to claim 30, wherein the protocol progress sensor detects an occlusion of a light source.
